(19) United States
(12) Patent Application Publication
(10) Pub. No.: US 2007/0250286 A1
(43) Pub. Date: Oct. 25, 2007

(54) Motion Monitoring and Analysis System

(75) Inventors: Michael Duncan, Sydney (AU); Simon Parker, Sydney (AU); Charles Worringham, Brisbane (AU); Peter Condie, Kelvin Grove (AU); Andrew Barriskill, Lane Cove (AU); Branka Curcic, Sydney (AU); Jan Jasiewicz, Brisbane (AU); Tony Parker, Brisbane (AU)

Correspondence Address: HARNESS, DICKEY & PIERCE, P.L.C., P.O. BOX 828, BLOOMFIELD HILLS, MI, 48303, US

(73) Assignee: Queensland University of Technology, Brisbane (AU)

(21) Appl. No.: 10/562,858

(22) PCT Filed: Jun. 25, 2004
(86) PCT No.: PCT/AU04/00839

§ 371 Date: Feb. 22, 2007

(30) Foreign Application Priority Data
Jul. 1, 2003 (AU) 20033903347
Apr. 16, 2004 (AU) 2004902053

(51) Int. Cl. A61B 5/103

(52) U.S. Cl. 702/139; 702/153

(57) ABSTRACT

A motion analysis system (10) in which sensor elements (20) attached to movable body segments record movement parameters including angular velocity and acceleration. A control device (40) receives the movement parameters and determines an overall motion of the movable body segments. The overall motion is analysed against an acceptable motion model to determine whether the overall motion is within acceptable limits. The sensor elements (20) and control device (40) are lightweight and can be worn during normal movement activities thereby allowing monitoring of work-based activities, such as lifting or typing. The invention is useful for detecting and correcting problems leading to lower back disorders and repetitive strain injuries.

### FIELD OF THE INVENTION

The present invention relates to a system for analysing, monitoring and recording the motion of a number of moving parts of a body performing a task or range of tasks. In particular, the present invention relates to a system for analysing, monitoring, and recording the motion of a human body during acute and chronic lifting tasks.

### BACKGROUND OF THE INVENTION

Workplace related injuries and in particular, back injuries, are significant problems in many industrialized countries. Such injuries are costly to a country's economy in terms of treatment for the injury as well as the loss of productivity that such injuries bring to the workplace.

For instance, in Australia, there are approximately 500,000 reported workplace related injuries reported each year, of which 25% are injuries to the lower back. In Queensland alone, it is estimated that injuries relating to manual handling, which are typically related to the condition referred to as low back disorder (LBD), resulted in the loss of over 40,000 workdays. Equally, in the United States it is estimated that costs associated with back injuries alone easily exceed US $40 billon. As such, many companies are finding that more and more of their expenses are being channelled into workers compensation payments and associated insurance than has traditionally been the case. In this regard, there is no surprise that conditions such as LBD have been identified as a major work place health and safety issue.

Another common workplace injury is repetitive strain injury (RSI). The term RSI has been used to describe many different types of soft tissue injury including carpel tunnel syndrome and tendinitis. It is usually caused by a mixture of bad ergonomics, poor posture, stress, and repetitive motion. The most common form of RSI in the workplace has been associated with computer usage however any form of repetitive motion can lead to soft tissue injury.

In order to prevent or decrease these injuries there is a need to investigate and evaluate factors associated with the mechanics of motion, such as lifting, that are shown to increase the incidence of workplace injuries such as LBD and RSI. It is considered that by quantifying such factors, it may be possible to lower the risk of injury to the worker.

Using LBD as an example, various methodologies exist that analyse lifting tasks in order to quantify LBD risks, such as the National Institute for Occupational Safety and Health (NIOSH), Static Strength Prediction Program (SSPP), Lumbar Motion Monitor Program (LMMP), United Auto Workers-General Motors Ergonomic Risk Factor Check List (UAW-GM RFC) and other less well known measures such as Rapid Upper Limb Assessment(RULA), Rapid Entire Body Assessment (REBA) and Manual Task Review and Assessment (MANTRA). Whilst each of these methodologies take a slightly different approach to assess LBD risk, they all take into account common factors such as weights lifted, starting heights, reach distances and posture.

The NIOSH equation is perhaps the most well known of these methodologies and is designed as a paper and pencil assessment tool for ergonomists or workplace assessment officers providing an empirical method for computing a weight limit for manual lifting. This limit has proven useful in identifying certain lifting jobs that posed a risk to the musculoskeletal system for developing lifting related low back pain.

One system for applying the NIOSH equation is described in U.S. Pat. No. 5,621,667. This patent describes an instrumented analysis system based on a retractable cable and potentiometer system which can determine the NIOSH equation multipliers indicative of physical parameters related to a lifting task under analysis. However, the instrumented system described is a rather dedicated system and is cumbersome and not easily implemented in a normal working environment. Further, such a system will not allow unimpeded long term analysis of the lifting task during the entire workday. In this regard, the system requires a dedicated space to set up the instrumentation and experienced personnel to operate the system and as such is better suited to a laboratory environment than a regular work environment.

Typically, there are very limited facilities to perform monitoring and analysis of factors associated with tasks such as lifting within the actual work environment. Workplace assessment, as it pertains to LBD, can only monitor motion for short periods of time or use equipment that is cumbersome to use in a regular work environment over prolonged periods of time, and takes time to analyse. In addition most of the workplace assessment tools, contain measurement of some subjective components which are difficult to accurately and reliably assess and are usually dependent on the experience of the assessor taking such measurements.

Whilst more detailed motion analysis can be achieved in laboratory settings using laboratory-based equipment, such as video motion analysis systems, these systems are expensive to construct and operate, and access to such facilities is typically restricted with there being only approximately 200 such facilities worldwide. In addition, laboratory analysis sessions require simulated work environments which in reality do not reflect the real work situation. In addition these facilities are only able to monitor motion in the laboratory setting for a short period of time during which a subject is being studied in the laboratory, and the ability to monitor motion over a longer period of time and in everyday workplace conditions, is not possible.

Therefore, the ability to monitor and analyse overall motion in the industrial or workplace setting, provides the opportunity to identify aspects of the overall movement that affect the functionality of the overall movement. When applied to human motion in the industrial or workplace setting, it may be possible to identify specific patterns of movement which increase the risk to worker injury, be it lifting or other repetitive movements. This may be particularly important for those involved in assessing workplace safety, such as ergonomists, workplace safety consultants, and health professionals who may wish to analyse the work environment including the motion of the worker within the environment in great detail so as to minimise injury and ensure maximum safety and optimise workplace efficiency. Equally, individuals may also wish to analyse motion of those who need to be trained to perform a particular task (i.e. lifting) in detail to ensure minimization of injury risk and to assess where help is needed.

In order to enable greater access to devices that can monitor human motion in the work environment throughout the day, a need therefore exists for a motion analysis system, which is low in initial cost, reliable, robust, simple and low cost to operate. Ideally such a system could be worn in the work environment by a subject/worker for extended periods allowing motion data to be collected under actual working conditions as the subject goes about their work activities.

In particular, such a system would make workplace analysis far more widely available for those individuals requiring such careful workplace monitoring and assessment incorporating many of the common assessment tools (including but not restricted to NIOSH, LMMP, SSPP, UAW-GM RFC or future assessment tool). In addition, the applications of such a system would be far broader. For example, such a system could be used to analyse real-time motions associated for training, modelling, real time monitoring, incorporation of physiological measures, work redesign and general injury prevention.

### SUMMARY OF THE INVENTION

The present invention resides in a system for monitoring motion of a subject, the system comprising:
a plurality of sensor elements mounted to movable body segments of a subject, said sensor elements capable of sensing parameters associated with individual movement of the body segments;
at least one control device for receiving said sensing parameters from said sensor elements and combining said sensing parameters to determine overall motion of said movable body segments; and
an analysis means for analysing said overall motion of said movable body segments to determine whether said overall motion of said movable body segments is within acceptable limits.

Preferably, the analysis means is a software program that is stored on said control device. Said analysis means compares parameters associated with said overall motion with parameters associated with a motion within safe and accepted limits and indicates whether the overall motion of the subject is within said safe and accepted limits.

The analysis means may also monitor accumulated load and provide an alarm if accumulated load exceeds an acceptable limit.

Alternatively, the system further comprises a remote computing device and the analysis means is a software program stored on said remote computing device.

In one form the control device is embedded in each sensor element. The plurality of control devices operates in a distributed fashion to determine overall motion. In this form the analysis means may be a remote computing device, such as a PC, programmed to compare parameters associated with the overall motion with parameters associated with a motion within safe and accepted limits. Alternatively the analysis means may be a portable computing device or a designated master sensor similarly programmed.

In another form the control device is a central control device in the form of a portable computing device which is suitably a body worn controller, such as a PDA or the like, which centrally receives said sensing parameters from the sensing elements and combines the sensing parameters to determine overall motion of said movable body segments. In this form the analysis means is suitably software programmed in the portable computing device. Alternatively the analysis means may be a remote computing device programmed with suitable analysis software.

In a still further form the control device and analysis means may be programmed in a remote computing device and each sensor includes a transmitter to transmit the sensing parameters to the remote computing device for determination of the overall motion and analysis of the motion.

In a yet further form, there is a control device embedded in each sensing element which communicates with a central control device or a designated master sensor. The central control device may be a portable computing device worn on the subject or a remote computing device remote from the subject. In this form the analysis means is software programmed in the central control device.

Preferably, the sensor element includes a data memory (which may be fixed or removable) and microprocessor for storing and processing said sensed parameters of movement.

Preferably, each sensor element includes at least one gyroscope and at least one accelerometer for measuring angular velocity and position of the movable body segment in at least one or more planes of motion and for measuring acceleration components. More preferably, each sensor element monitors angular velocities in the sagittal, coronal and transverse planes of the body segment that the particular sensor element is mounted on and monitors linear and angular acceleration experienced in three dimensions (radial, tangential and centripetal) in relation to the body segment to which it is attached.

Each sensor preferably also includes a magnetometer for determining absolute position in the horizontal plane.

Preferably, each sensor element may also monitor the sagittal, coronal and transverse angles of its associated body segment. Each sensor element may also measure pressures as a resistance measurement from pressure sensors provided with the system. Further, each sensor element may also measure strain via strain gauges provided with the sensor element. The sensor also preferably has the provision to accept external signals from other devices such as, but not restricted to heart rate monitors, other physiological measurement devices, instrumented shoes (for example, pressure distribution systems), miniature lumbar or spine motion monitors and fixed force plates.

To facilitate operation of the control device, the control device preferably has a display screen. The display screen may be a liquid crystal display (LCD) that may be used for controlling the operation of the system. Further, the LCD may be used by an operator for analysis and display of the motion data obtained by the motion analysis system. The control devices can also be used to program the embedded sensor control device in order to perform general and specific functions via hardwire or wireless communication protocols.

The control device may include a memory card slot such as a flash emory card slot. Note that a memory card can be also embedded within sensor.

The system may further comprise an interface unit that facilitates bi-directional communication between the central control device and the sensor elements.

The interface unit may include a remote control interface. This may be a bi-directional communications interface between a remote control unit and a control device. Also, this communications interface may be a wireless interface so as to minimise the necessity for fitment of cables and removing the possibility of cables becoming entangled or dislodged.

Alternatively, the control device may include a remote control facility that enables the operator to interact with the system remotely without the need for physically operating the control device. Preferably, a remote control unit provides a clinician with a non-visual confirmation of communication of data from the remote control unit to the control device.

The invention also resides in a method of monitoring motion of a subject including the steps of:
sensing parameters associated with individual movement of body segments;
combining said sensing parameters to determine overall motion of said body segments;
analysing said overall motion to determine if said motion is within acceptable limits; and
indicating whether said overall motion is within said acceptable limits.

The method may further include the step of recording said overall motion for later analysis.

The method may also include the step of monitoring accumulated load and providing an alarm if accumulated load exceeds an acceptable limit.

### BRIEF DESCRIPTION OF THE DRAWINGS

The invention is now described by way of example with reference to the accompanying diagrammatic drawings in which:

FIG. 1 shows a motion monitoring and analysis system for monitoring a lifting motion of a subject;

FIG. 2 shows a block schematic diagram of motion monitoring and analysis system;

FIG. 3 shows a block schematic diagram of a sensor element;

FIG. 4 shows a software screen associated with the present invention that displays a NIOSH analysis of a lifting task; and

FIG. 5 shows an interactive frame-by-frame analysis screen of a lifting task.

### DETAILED DESCRIPTION OF THE DRAWINGS

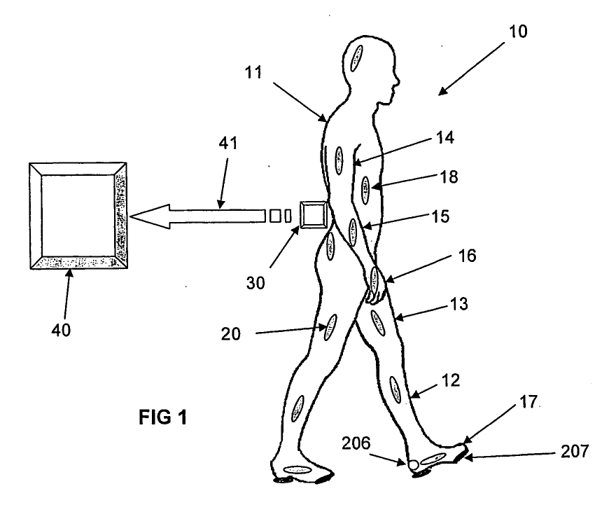

The motion monitoring and analysis system of the present invention is generally referred to by reference numeral 10 in FIG. 1. The system 10 includes a plurality of sensor elements, such as sensor element 20, removably attached to segments of the body I1 which require monitoring. For instance, to monitor lifting motion sensor elements 20 may be attached to the lower leg 12, upper leg 13, upper arm 14, lower arm 15, hand 16, foot 17 and torso 18. Each of the sensor elements 20 are provided with associated componentry and circuitry to measure, process and store motion data of the body segment upon which it is placed. Each of the sensor elements 20 communicate with a controller 30 via a wired (not shown) or wireless connection to feed the motion data obtained by the sensor elements for further processing. The control device 30 is a body worn or carried device which can further communicate with a remote computing device 40, such as a remotely positioned personal computer. The remote computing device 40 thereby providing remote access to the data collected and stored by the system 10.

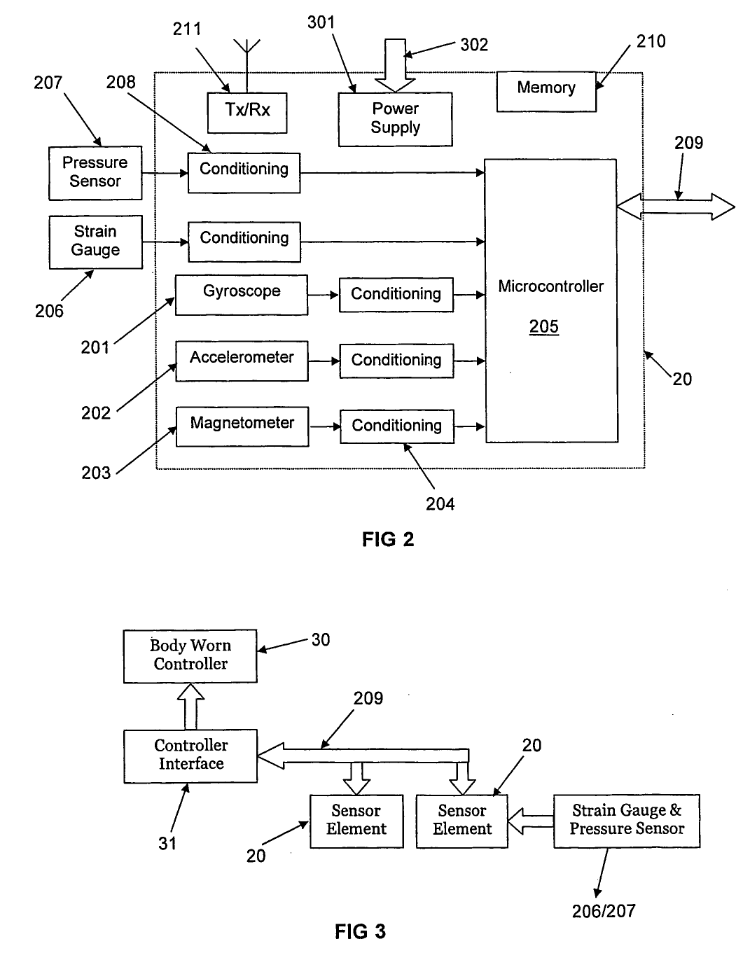

A schematic of a sensor element is shown in FIG. 2. Each of the sensor elements 20 consist of a small light weight casing and typically weigh less than 22 grams. The sensor elements are typically made from a plastic housing having a substantially rectangular shape with a length of 65 mm, a width of 34 mm and a thickness of 12 mm. The dimensions of the sensor elements 20 are sufficient to house one or more gyroscopes 201 and one or more accelerometers 202 and a magnetometer 203. The gyroscope 201 communicates with a microprocessor 205 via signal conditioning circuitry 204. Similarly there is signal conditioning circuitry associated with the accelerometers 202 and the magnetometer 204.

Each sensor element 20 may also receive signals from external sensors such as strain gauges 206 or pressure sensors 207. For example, when monitoring lifting motion there may be a heel pressure sensor and a ball pressure sensor to monitor shift of weight during lifting. The pressure sensors 207 and strain gauges 206 communicate with the control device 30 via at least one of the sensors 20. For this purpose, each sensor 20 includes general purpose signal conditioning inputs 208. The inputs 208 are suitably analogue inputs.

Each sensor 20 communicates with the control device 30 via a communications bus 209, as shown in FIG. 3. The communications bus 209 is a serial peripheral interface (SPI) bus.

The sensor 20 also receives power from the control device 30 via the SPI bus as indicated at 302. The power 302 is fed to the microprocessor 205 of the sensor 20 via a power supply 301, which may be a regulator circuit which includes a brownout/reset circuit.

Each sensor element 20 is capable of measuring angular velocity of the body segment in at least one or more planes of motion, measuring acceleration components in three dimensions and measuring outputs from pressure sensors 207 and strain gauges 206 or three pressure sensors alone for determining positional information of the lower extremities, upper extremities, torso and head of the subject's body. More particularly, each sensor element 20 monitors angular velocity in the sagittal plane of the body segment that the particular sensor element 20 is mounted on. Further, the sensor element 20 monitors linear acceleration experienced in three dimensions in relation to the body segment to which it is attached. Each sensor element 20 can also monitor the sagittal, coronal and relative transverse angles of its associated body segment.

Each sensor element 20 may accept signal inputs from peripheral devices such as, simple pressure sensitive transducers (i.e. on-off switches), heart rate monitors, foot pressure distribution systems, global positioning sensors, portable gas analysis systems or other devices in order to incorporate this information with body movement. A useful peripheral device is a microphone so that a subject can verbally input an index of perceived effort. This is usually a simple numerical index with larger numbers indicating greater perceived effort. Suitable voice recognition software translates the spoken number to a signal for use in the analysis software.

The specific function of a sensor element 20 will be dictated by the combination of sensors. If it is only desired that a particular sensor measure relative motion in two dimensions than only a pair of gyroscopes is required. Adding a third gyroscope allows relative motion in three dimensions to be measured. In order to measure absolute position an accelerometer is required to determine vertical position relative to gravity. To determine absolute position in the horizontal plane a magnetometer is also required.

The data collected by each sensor element 20 may be stored in the on-board memory of the microprocessor 205 or separate memory device 210 and downloaded to the control device 30 at a designated time delay following data collection or upon request from the control device 30. The memory device 210 may be a fixed storage device or a removable device such as a flash memory stick. The raw sensor data may be stored in the removable memory device for later retrieval and detailed off-line analysis. This may be useful in the case where only processed movement data is sent from a sensor element 20 to the control device 30. A greater level of analysis may be possible with the raw data.

In another embodiment data is collected in real time by the control device 30. The data may be processed data or raw data, but will usually be processed data that defines the movement of the body segment rather tan the raw data from the accelerometer, magnetometer and gyroscopes. In this regard, the control device 30 constantly receives the information from each sensor element 20 during the motion. In certain embodiments the sensor element 20 may include a low power transmitter/receiver 211 for communication with a control device 30 or remote computing device 40.

In a preferred embodiment, each sensor element may contain an embedded and programmable control device incorporating a memory unit without the need for an external control device. Such an arrangement allows multiple sensors to operate independently in a distributed fashion wherein motion data can be downloaded for analysis at a later time. In such an embodiment, each independent sensor has a timing device, which can be synchronised to other sensors so that all data from multiple sensors can be fully synchronised. The sensor can also be connected to other sensors and an external control unit using wireless communication, such as but not limited to “bluetooth” technology, via the transmitter/receiver 211.

A variation on this embodiment utilises wireless access point technology in a workplace. Each wireless access point defines a zone, referred to as Hot Spot zones, in which data may be retrieved from the control device and/or sensor elements. When a subject enters the zone a communication channel is opened to automatically transfer data for further analysis. Instructions can also be transmitted to the control device and sensor elements.

As mentioned previously, there is suitably at least one accelerometer 202. This is suitably a 3D accelerometer but could be two 2D accelerometers. Each 2D accelerometer measures in two dimensions but combine to measure all three dimensions of acceleration. Each accelerometer measures two of the three dimensions of acceleration with one of the accelerometers being oriented perpendicular to the PCB so that the linear acceleration in the sagittal and coronal planes may be measured. Thus, one dimension may be measured twice to provide a reference and confirmation of the measurement. Also the particular direction of movement with respect to gravity can be determined. Each accelerometer produces two analogue voltage outputs corresponding to two dimensions of linear acceleration. It also produces a pulse width modulated output corresponding to the two dimensions of linear acceleration. A self test facility is also included wherein the self test signal is taken from a digital output of the micro-controller 205. Specific suitable accelerometers that can be used are ADXL 202. A power cycling operation can be used by switching the power supply off in order to save power.

In a system where lift task monitoring is desired, it will be appreciated that, in respect of each of the lower extremities, the relative positions of the thigh (the part of the lower extremity between the hip and knee), leg (the part of the lower extremity between the knee and ankle) and arms (the part between the forearm and upper arm) relative to each other and the torso need to be monitored. Accordingly, each upper and lower extremity may use at least six sensor elements 20, one mounted on the thigh, one mounted on the leg, one mounted on the foot, one mounted on the torso, one mounted on the forearm and one mounted on the upper arm, for a total of twelve sensors. However it should be appreciated that the position and number of sensor elements 20 employed can vary depending on the type of motion to be analysed.

In essence, each sensor element 20 is capable of recording detailed measurement of a number of aspects of motion of the body segment upon which it is mounted. Whilst the number and types of measurements that can be taken by each pressure sensor 20 is considerable in light of prior art systems, each pressure sensor is housed in a small and robust housing that can easily be worn on each body segment without limiting the movement of that body segment. This enables an individual to attach a number of sensors onto desired body segments and to wear such sensors under clothing and perform work related activities during the day, such that their continual motion including specific work related tasks, such as lifting can be monitored and analysed, without adversely affecting their movement.

As shown in FIG. 3, the data from the sensor elements 20 is fed via an interface unit 31 to the control device 30. The control device 30 is worn externally of the subject's body, for example in a pocket or pouch located on the subject. The control device 30 may be in the form of a hand-held programmable device. Preferably, the control device 30 is in the form of a commercially available portable computing device, such as a pocket PC, PDA or a PDA-mobile telephone combination device. In this case the interface unit 31 is suitably a flash memory interface that interfaces to the control device 30 via a compact flash bus. The interface unit 31 facilitates bi-directional communication with the sensor elements 20.

The control device 30 includes a display, which for the portable computing device is in the form of a liquid crystal display (LCD). Preferably, the LCD is implemented in the form of a touch-sensitive screen for enabling a workplace assessment officer or ergonomist to select, via appropriate icons on the screen, the motion analysis to be effected.

The interface unit 31 may include a peripherals interface in the form of a queued serial peripheral interface (QSPI) for interfacing with the sensor elements 20. It is envisaged that the control device 30 can communicate with the/or each of the sensor elements 20 via a conventional wire link or via a wireless link. A wireless link will enable a greater amount of freedom of movement and ease of use to enable the subject or worker to perform the desired working tasks with minimum impedance.

A remote control interface 41 is included for communicating with the remote computing device 40 of the system 10. The remote computing device 40 may be a PC located remotely from the worker or subject of interest, for example, the remote computing device 40 may be a PC located and operated by an ergonomist or workplace safety officer situated in an area remote from the subject. In this regard, the sensed motion of the subject or worker can be monitored as they perform common tasks such as lifting objects, and the various measurements taken from the sensor elements 20 can be sent to the remote computing device for analysis. This could occur in real-time such that a worker's motion can be monitored in order to ensure that the worker uses safe practises, such as manner of lifting, and to detect or monitor cumulative loads before the onset of a problem, such as LBD, or may occur following completion of a task or at the end of a designated time period, such as at the end of each day.

The system of the present invention can be used to alert a worker, using an alarm, that their current motion is not optimal, upon detection of a specified criterion being exceeded during a performed task. Such a criterion can be calculated by applying common work place safety assessment tools, such as the NIOSH equation, or other similar measures previously mentioned using the collected data. As a result, the present system can be used in a number of applications, which have previously not been possible due to the fact that the subject was confined to laboratory spaces and dedicated laboratory equipment.

The present invention can easily be applied to monitoring manual lifting tasks and calculating the NIOSH Lifting Index (LI) for a worker or subject performing their daily work routine. As alluded to previously, the NIOSH equation uses 5 risk factors to calculate a recommended weight limit (RWL) for lifting. NIOSH starts with a 23 kg load constant that is reduced by a multiplier for each risk factor that has a value of less than 1. Multipliers are computed for the horizontal distance between the part lifted and the body, the start height, the vertical distance lifted, lift asymmetry, the quality of the hand object interface (coupling) and the frequency of lifting. The LI is computed by dividing the weight of the object to be lifted by the RWL. Lifting tasks that have a LI of less the 1 are considered acceptable. LI ratios of 1 or greater will put a healthy worker at increased risk for LBD. The NIOSH equation also has provision to consider physiological factors such as energy expenditure, but until now it has been difficult to incorporate such measures. It can be immediately appreciated that this invention will allow measurement of physiological measures during lift, which is a significant advance over the prior art.

The NIOSH analysis program of the present invention is comprised of two programming elements. The first programming element is deployed on the body worn controller, typically a PDA. The second programming element is deployed on the remote computing device 40, typically a PC. With regard to the first processing element, the sensor data is processed by the PDA in real time, basic analyses is performed and the raw data is stored and processed. With regard to the second processing element, the PC program enables more detailed analyses of the lifting tasks, such as modeling and performing “what if” scenarios for work place redesign and other emerging applications once the data has been downloaded from the PDA. In this regard, such complex analyses can be done at a later time. In addition, the deployment of analysis software on a PC allows creation of a database of results so as to monitor progress and record keeping.

The purpose of the PDA analysis program is to convert raw sensor data obtained from the sensor elements 20 and calculate common parameters (both kinematic and spatiotemporal). The basic concept includes converting segmental tilt angles into segment coordinates that are then used to reconstruct an animated biomechanical stick figure using trigonometry or other mathematical conversion methods (spherical coordinates). The biomechanical segmental model depends on the number of sensor elements 20 that are attached to the subject. If seven sensor elements 20 are used it will create a seven-segment model. This allows accurate determination of user specified events during manual handling tasks such as when the worker picks up the object to be lifted and when the worker lets go at the destination. The determination of such events can then be used to calculate the initial height, the horizontal distance of the object from the worker, the distance of the lift, the quality of coupling, lift frequency and the asymmetry of the lift (amount of twisting of the torso), all of which are important factors to consider to calculate the NIOSH lift index. The weight of the object to be lifted can also be obtained by an operator entering in such a value to the system, although a provision exists to determine the weight automatically using a small scale or knowledge of the goods being handled. From this the PDA is able to calculate the NIOSH lift index of a single lift, the cumulative load above and below the criterion LI of 1 from multiple lifts and provides auditory feedback or alarm if an LI of 1 is exceeded during an actual lift. In addition, the frequency and/or amplitude of the warning tone changes proportionally to increasing LI values (>1).

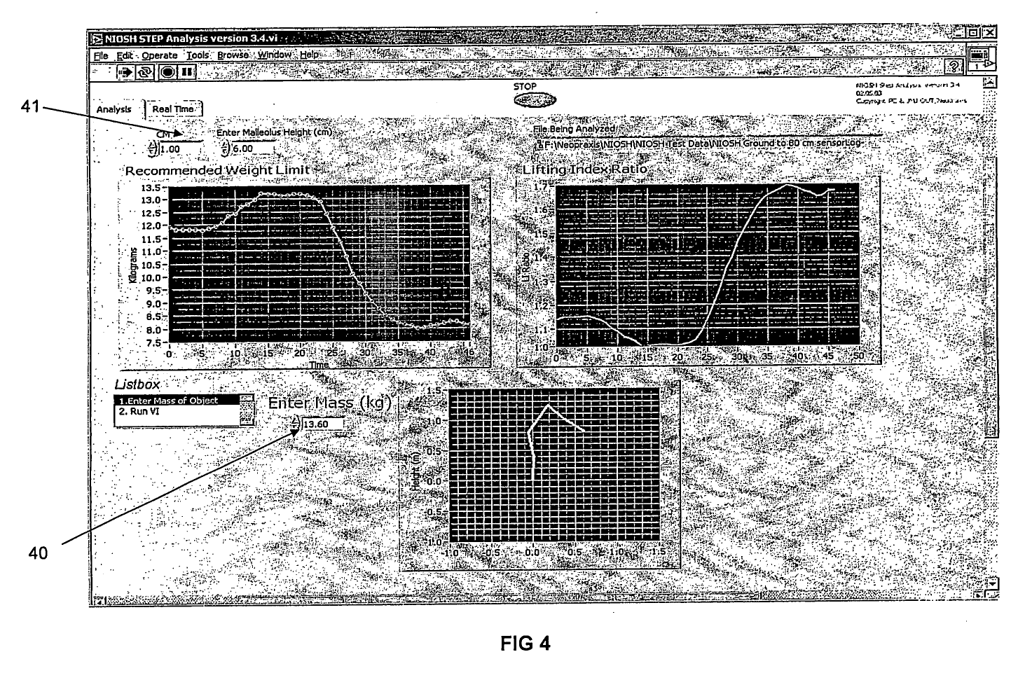

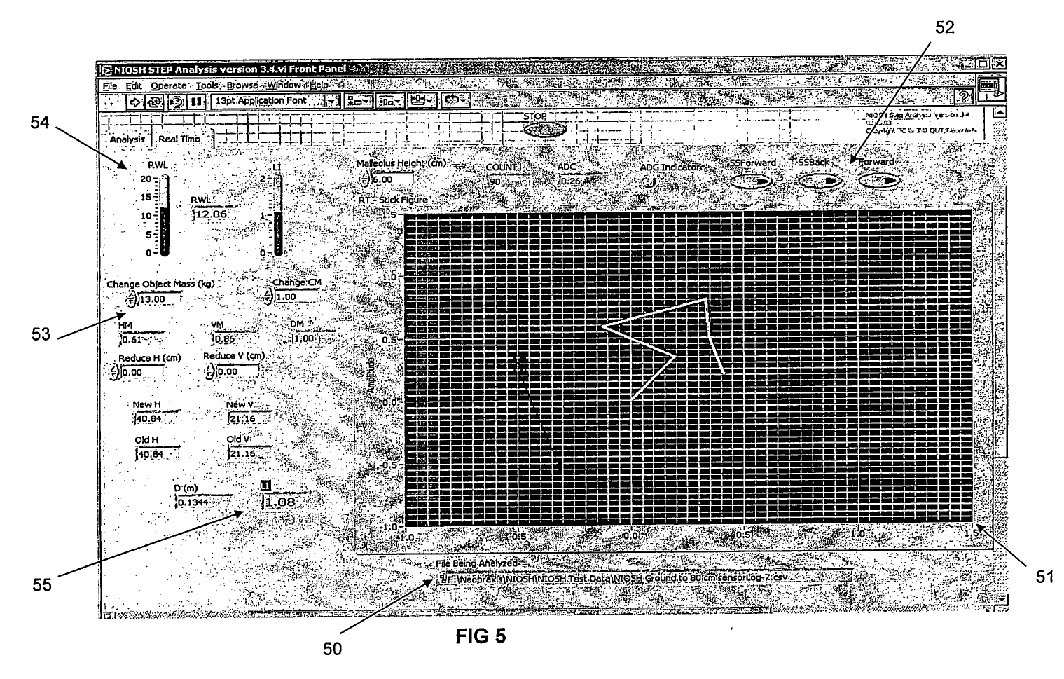

FIG. 4 shows the analysis screen of the NIOSH analyser program. Data is firstly downloaded from the control device 30. When the program is executed the anthropometric date originally entered into the control device is read. From the data the X-Y coordinates and rotation angles of the body segments are calculated. This data is then used to calculate the relevant parameters for the NIOSH equation.

A user enters the object weight 40 that was lifted and a coupling multiplier (CM) 41. The screen graphically displays the spatiotemporal parameters calculated for movements of all limbs and animates the actual lift. The results can be saved as a spreadsheet file. The bottom panel displays the motion being analysed. The ability to obtain such important information via such a discrete and easy to use system as the present invention provides advantages not before realizable with prior art motion analysis systems.

FIG. 5 shows an interactive frame-by-frame analysis screen in which lift parameters can be manipulated to reduce the lift task index. This is particularly useful in redesigning the workplace or the task. The file being analysed is shown 50 as well as a stick figure animation 51 of the motion under analysis. The stick figure representation of the sensor data during the lifting task is fully animated and controllable. The user controls the movement of the stick figure by clicking the appropriate frame-by frame button 52 or in normal time mode. As is shown, the controls are similar to VCR controls. The user animates the stick figure frame by frame and when a specific event (i.e. when the object is grabbed by the worker) is identified, the user will enter the appropriate data 53. The program also allows for automatic event identification for many trials in which the worker lifts many objects during the course of the work episode. This facilitates rapid analysis and automatically calculates lift frequency (lifts/minute). The user can also review identified events and if necessary corrections can be made. This feature allows an operator to determine which parameter has the greatest effect in reducing the Lift Index. The results of changing the NIOSH parameters are shown by index bars 54 and display fields 55. As can be appreciated, this is particularly useful in workplace or task redesign and for lift training. If no event signals were collected event identification is done manually.

Once events are identified, the program extracts the data between events, normalizes it and calculates the relevant NIOSH equations. All data are saved for further presentation, or saved into a database.

In this regard, the present invention lends itself to a number of commercial uses. One obvious adaptation of the present invention is in monitoring and analysing worker motion as to train and inform workers about how to perform a specific task or tasks as safely as possible under varying task conditions. This can be done by providing instantaneous feedback or by visually comparing incorrect movements with that of correct movement. The present invention can also be adapted to be used by the ergonomist to design an optimum work area.

Other applications of the present invention may be in monitoring and analysing the motion of workers in order to allow for work place redesign so as to modify the task and the work place environment to minimize risk to the worker(s). This will have additional benefits because it will permit the minimization of work place injury (injury prevention) and increase production efficiency. It can be readily appreciated that the cost of preventing injury is of great benefit to the individual worker, society and the company. As mentioned, this can occur in real time situations, something which has not been possible with prior art systems.

Many of the common tools used to assess the risk of workplace injury are limited by shortcomings in monitoring systems. This is because until now it was not possible to measure human motion under actual working conditions. The present invention permits important advances in the measurement of workplace injury risk, either by refinement of existing measures or the development of entirely new assessment tools. For instance the system will allow for tests to be developed that take into account specifics of the individual worker, such as the worker's physiological capacity, age and gender as well as previous history of injury. Whilst the above applications are only examples of the commercial applications of the present invention, it is envisaged that there are many more examples that equally apply.

Throughout the specification the aim has been to describe the invention without limiting the invention to any one embodiment or specific collection of features. Persons skilled in the relevant art may realize variations from the specific embodiments that will nonetheless fall within the scope of the invention.

1. A system for monitoring motion of a subject, the system comprising:
a plurality of sensor elements mounted to movable body segments of a subject, said sensor elements capable of sensing parameters associated with individual movement of the body segments;
at least one control device for receiving said sensing parameters from said sensor elements and combining said sensing parameters to determine overall motion of said movable body segments; and
an analysis means for analysing said overall motion of said movable body segments to determine whether said overall motion of said movable body segments is within acceptable limits.

2. The system of claim 1 wherein the analysis means is a software program that is stored on said control device.

3. The system of claim 1 wherein the analysis means also monitors accumulated load and provides an alarm if accumulated load exceeds an acceptable limit.

4. The system of claim 1 wherein the analysis means compares parameters associated with said overall motion with parameters associated with a motion within safe and accepted limits and indicates whether the overall motion of the subject is within said safe and accepted limits.

5. The system of claim 1 further comprising a remote computing device wherein the analysis means is a software program stored on said remote computing device.

6. The system of claim 1 comprising a control device embedded in each said sensor element.

7. The system of claim 6 wherein the analysis means comprises a remote computing device programmed to compare parameters associated with the overall motion with parameters associated with a motion within safe and accepted limits.

8. The system of claim 6 wherein the analysis means comprises a portable computing device programmed to compare parameters associated with the overall motion with parameters associated with a motion within safe and accepted limits.

9. The system of claim 1 wherein the control device is a central control device in the form of a portable computing device which centrally receives said sensing parameters from the sensing elements and combines the sensing parameters to determine overall motion of said movable body segments.

10. The system of claim 9 wherein the analysis means is software programmed in the portable computing device.

11. The system of claim 1 further wherein the analysis means is a remote computing device programmed to compare parameters associated with the overall motion with parameters associated with a motion within safe and accepted limits.

12. The system of claim 1 further comprising a transmitter means associated with each said sensor element.

13. The system of claim 12 further comprising a remote computing device, the at least one control device and the analysis means are programmed in said remote computing device and each sensor transmits said sensing parameters to the remote computing device for determination of the overall motion and analysis of the motion.

14. The system of claim 6 further comprising a central control device worn on the subject and wherein the analysis means is software programmed in the central control device.

15. The system of claim 6 further comprising a remote computing device remote from the subject wherein the analysis means is software programmed in the remote computing device.

16. The system of claim 1 wherein the sensor element includes a data memory and microprocessor for storing and processing said sensed parameters.

17. The system of claim 1 wherein each sensor element includes at least one gyroscope and at least one accelerometer for measuring angular velocity of the movable body segment in at least one or more planes of motion and for measuring acceleration components.

18. The system of claim 1 wherein each sensor element includes a magnetometer.

19. The system of claim 1 wherein each sensor element includes at least one gyroscope, at least one accelerometer and at least one magnetometer and measures absolute motion and position in three dimensions.

20. The system of claim 1 wherein each sensor element includes one or more signal conditioning means.

21. The system of claim 1 wherein each sensor element includes one or more external sensor inputs.

22. The system of claim 1 wherein each control device has a display screen.

23. The system of claim 22 wherein the display screen is a touch used by an operator for analysis and display of motion data obtained by the system.

24. The system of claim 1 wherein the control device includes a memory card slot.

25. The system of claim 1 further comprising an interface unit that facilitates bi-directional communication between said one or more control devices and said plurality of sensor elements.

26. The system of claim 25 wherein interface unit includes a remote control interface.

27. The system of claim 1 wherein the control device includes a remote control facility that enables an operator to interact with the system remotely without the need for physically operating the control device.

28. A method of monitoring motion of a subject including the steps of:
sensing parameters associated with individual movement of one or more body segments;
combining said sensing parameters to determine overall motion of said body segments;
analysing said overall motion to determine if said motion is within acceptable limits; and
indicating whether said overall motion is within said acceptable limits.

29. The method of claim 28 further including the step of recording said overall motion for later analysis.

30. The method of claim 28 wherein said parameters include one or more of angular velocities in the sagittal, coronal and transverse planes of said body segments and linear acceleration experienced in three dimensions in relation to said body segments.

31. The method of claim 28 wherein said parameters include one or more of sagittal, coronal and relative transverse angles of said body segments.

32. The method of claim 28 further including the step of measuring pressure as a resistance measurement from pressure sensors associated with at least one of said one or more body segments.

33. The method of claim 28 further including the step of measuring strain via strain gauges associated with at least one of said one or more body segments.

34. The method of claim 28 further including the step of measuring one or more other parameters from devices including heart rate monitors, other physiological measurement devices, instrumented shoes and fixed force plates.

35. The method of claim 28 further including the step of the analysis means monitoring accumulated load and providing an alarm if accumulated load exceeds an acceptable limit.
